(19) United States
(12) Patent Application Publication
(10) Pub. No.: US 2019/0357917 A1
(43) Pub. Date: Nov. 28, 2019

(54) JUNCTIONAL HEMORRHAGE CONTROL DEVICE

(71) Applicants: Philip M. Chun, San Diego, CA (US); Daniel G. Terry, Pleasant Hill, CA (US); Jeffrey R. Dempsey, Spring, TX (US)

(72) Inventors: Philip M. Chun, San Diego, CA (US); Daniel G. Terry, Pleasant Hill, CA (US); Jeffrey R. Dempsey, Spring, TX (US)

(21) Appl. No.: 16/485,413

(22) PCT Filed: Feb. 13, 2018
(86) PCT No.: PCT/US18/18060

§ 371 Date: Aug. 12, 2019

Related U.S. Application Data
(60) Provisional application No. 62/460,427, filed on Feb. 17, 2017.

(51) Int. Cl. A61B 17/132

(52) U.S. Cl. CPC A61B 17/1325; A61B 2017/12004

(57) ABSTRACT

Provided herein is a junctional hemorrhage control device that can be used with a compressing or constriction device so as to provide improved control of blood flow through arteries and veins of a subject for a period of time when the compressing or constriction device is tightened.

### CROSS REFERENCE TO RELATED APPLICATIONS

This application claims priority to U.S. Provisional Application Ser. No. 62/460,427, filed Feb. 17, 2017, the disclosure of which is incorporated herein by reference.

### TECHNICAL FIELD

Provided herein is a junctional hemorrhage control device that can be used with a compressing or constriction device so as to provide improved control of blood flow through arteries and veins of a subject for a period of time when the compressing or constriction device is tightened.

### BACKGROUND

Constricting or compressing devices, such as tourniquets, are used to control venous and arterial circulation to an extremity for a period of time. Pressure is applied circumferentially upon the skin and underlying tissues of a limb; this pressure is transferred to the walls of vessels, causing them to become temporarily occluded. These constricting or compressing devices are generally used as a tool for a medical professional in applications such as cannulation or to stem the flow of traumatic bleeding, especially by military medics. Moreover, these constricting or compressing devices are usually applied when the patient is in a life-threatening state as a result of continuous bleeding.

### SUMMARY

Provided herein is a junctional hemorrhage control device that can be used with a compressing or constriction device so as to provide improved control of blood flow through arteries and veins of a subject for a period of time when the compressing or constriction device is tightened. In particular, it was found that use of the junctional hemorrhage control device disclosed herein with a compressing/constricting device provided for better control of blood flow from the femoral artery and vein of a subject than use of the tourniquet alone. Moreover, due to the low weight, compact size, and ease of use, the junctional hemorrhage control device disclosed herein is superior to other devices which may have similar functionality but are much bulkier and not so easy to use.

In a particular embodiment, a junctional hemorrhage control device is provided herein that comprises: a protrusion portion that has a generally rounded upper surface and a flat bottom lower surface; a barrier portion comprising a left portion and a right portion, wherein the left portion and the right portion have the same geometries and dimensions; a base portion, wherein the flat bottom surface of the protrusion portion is in physical contact and attached to the base portion, and wherein the highest point of curvature for the upper surface of the protrusion portion is located above the center point of the base portion, and wherein the left portion and right portion of the barrier portion are in physical contact and attached to opposite sides of the lower surface of the base portion so as extend from opposite ends of the base portion, wherein between the left portion and right portion is a rectangular space; and a dry friction coating or material that is physically in contact with the central lower surface of the base portion and located in the rectangular space between the left portion and right portion of the barrier portion; wherein the junctional hemorrhage control device is configured to be used with a compressing or constricting device to control the blood flow of an artery and/or vein in an extremity of a subject. In another embodiment, a cross-section section of the protrusion portion has an arch-like shape. Examples of arch-like shapes, include one-centered (semicircular), two-centered, three-centered, four-centered, segmental, pointed segmental pseudo three-centered, or pseudo four-centered. In yet another embodiment, the edges of the protrusion portion are congruent with the edges of the base portion. In a further embodiment, the edges of the protrusion portion are not congruent with the edges of the base portion, wherein the distance between the outer edge of base portion to the outer edge of protrusion portion is from 0.05 to 0.5 inches. In yet a further embodiment, the highest point of curvature from lower surface of the protrusion portion is from 0.5 to 1.125 inches. In a certain embodiment, the protrusion portion is permanently attached to the base portion. In an alternate embodiment, the protrusion portion is reversibly attached to the base portion, and wherein the protrusion portion and base portion comprise an inner open space so that when protrusion portion and base portion are attached, the junctional hemorrhage control device is hollow. In another embodiment, the base portion has a diameter or length from 2.5 to 4 inches, and a height of 0.5 to 1.2 inches. In yet another embodiment, the base portion has a cylindrical shape, and the left portion and right portion of the barrier portion have segmented shapes. In a further embodiment, the width of the left and right portions of the barrier portion is from 0.15 to 0.4 inches. In yet a further embodiment, the base portion has a diameter of about 2.625 inches or about 3 inches and the width of the left and right portion of the barrier portion is about 0.25 inches or about 0.375 inches. In an alternate embodiment, the dry friction coating or material is a non-slip, anti-slip, or slip resistant-coating material that is applied to the surface of the base portion. In another embodiment, the left portion and the right portion of the barrier portion further comprises notches; and wherein the junctional hemorrhage control device further comprises a latch that can slideably fit within the notches of the left and right portions and that can be fixed in place by using a fixing means, and which can be released from the notches by using an opening means.

In a certain embodiment, the disclosure also provides a method of using a junctional hemorrhage control device disclosed herein, comprising: contacting the protrusion portion of the junctional hemorrhage control device with an extremity of a subject, wherein the junctional hemorrhage control device is placed over an artery and/or vein; affixing a compressing or constriction device around the extremity and in contact with the dry friction coating or material on the base portion of the junctional hemorrhage control device; and tightening the compressing or constriction device so as to control the blood flow through the artery and or vein of the extremity of the subject. In a further embodiment, the junctional hemorrhage control device is placed over the femoral artery and vein of the thigh portion of the lower extremity of the subject. In yet a further embodiment, the compressing or constriction device is a tourniquet.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1 provides diagrams of major arteries found in the upper and lower extremities.

FIG. 2 provides an MRI photo and diagram of the upper thigh region. The location of the femoral artery and vein is indicated.

FIG. 3 provides a head-on side view of an embodiment of a junctional hemorrhage control device of the disclosure.

FIG. 4 provides a head-on side view of an additional embodiment of a junctional hemorrhage control device of the disclosure.

FIG. 5 provides a bottoms-up view of an embodiment of a junctional hemorrhage control device of the disclosure.

FIG. 6 provides two three-dimensional side angled views of an embodiment of a junctional hemorrhage control device of the disclosure.

FIG. 7 provides a bottoms-up view of an alternate embodiment of a junctional hemorrhage control device of the disclosure.

FIG. 8 provides a head-on side view of an alternate embodiment of a junctional hemorrhage control device of the disclosure.

FIG. 9 provides a head-on side view (top) and top down view (bottom) of an alternate embodiment of a junctional hemorrhage control device of the disclosure.

FIG. 10 provides a head-on side view (top) and top down view (bottom) of an alternate embodiment of a junctional hemorrhage control device of the disclosure.

FIG. 11 provides two head-on side views of an alternate embodiment of a junctional hemorrhage control device of the disclosure that further comprises a latch and hinge, wherein the latch can be opened (top figure) and closed (bottom figure).

FIG. 12 provides a head-on side view of an embodiment of a junctional hemorrhage control device of the disclosure being used with a compressing or constricting device.

FIG. 13 provides a head-on side view of an embodiment of a junctional hemorrhage control device of the disclosure being used with a compressing or constricting device.

FIG. 14 provides an angled side view an embodiment of a junctional hemorrhage control device of the disclosure being used with a compressing or constricting device to put pressure on the femoral artery and vein of a subject's leg.

FIG. 15 provides an angled top down photo of an embodiment of a junctional hemorrhage control device of the disclosure being used with to put pressure on the femoral artery and vein of a subject's leg.

FIG. 16 provides a top down photo of an embodiment of a junctional hemorrhage control device of the disclosure being used with a tourniquet in an around the hip placement, to put pressure on the femoral artery and vein of a subject's groin.

FIG. 17 provides a side photo of an embodiment of a junctional hemorrhage control device of the disclosure being used with a tourniquet in a through a belt loop placement, to put pressure on the femoral artery and vein of a subject's leg. The tourniquet strap being passed through a belt loop is indicated by a circle.

FIG. 18 provides another side photo of an embodiment of a junctional hemorrhage control device of the disclosure being used with a tourniquet in a through a belt loop placement, to put pressure on the femoral artery and vein of a subject's leg. The tourniquet strap being passed through a belt loop is indicated by a circle.

FIG. 19 provides a top down photo of an embodiment of a junctional hemorrhage control device of the disclosure being used with a tourniquet in a through a belt loop placement, to put pressure on the femoral artery and vein of a subject's leg.

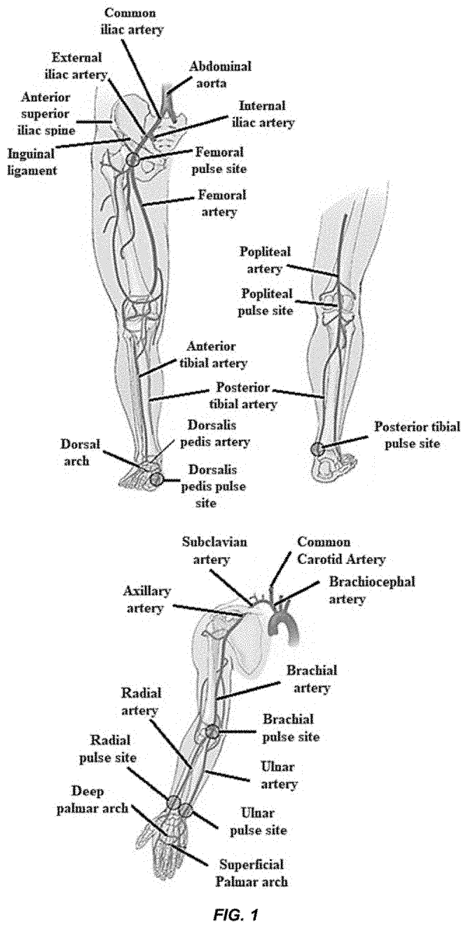

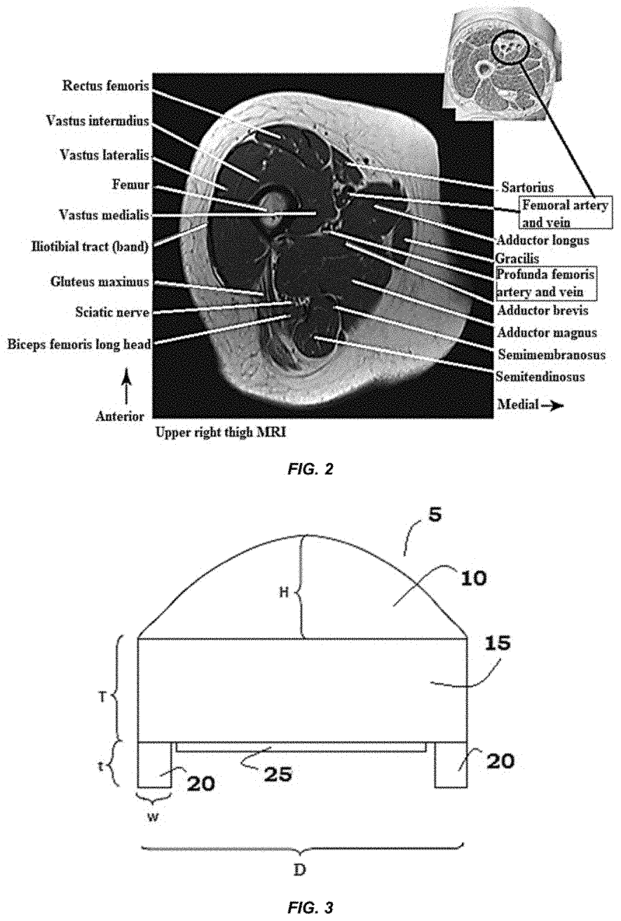

### DETAILED DESCRIPTION

As used herein and in the appended claims, the singular forms “a,” “an,” and “the” include plural referents unless the context clearly dictates otherwise. Thus, for example, reference to “a tourniquet” includes a plurality of such tourniquets and reference to “the constricting device” includes reference to one or more constricting devices, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

During the war on terrorism, military medicine has shifted its priorities to better save lives. Although the priority in civilian medicine is to treat the airway first, military medicine has shifted its priority to first treat massive hemorrhage, since it is the leading cause of death on the battlefield. Additionally, another reason to prioritize massive hemorrhage over treating the airway is because, on average, three minutes of uncontrolled massive hemorrhage leads to a battlefield death whereas five minutes of an unsecured airway leads to a battlefield death.

Currently, the military uses/provides fast acting, self-application tourniquets such as CAT (combat application tourniquet) and SOF T (special operations forces tourniquet) to stop massive hemorrhaging. These combat tourniquets are simple/fast acting devices used to stop bleeding on an extremity.

Despite the use of and availability of the tourniquets, helmet and body armor, the number of battlefield casualties continues to grow exponentially because of Improvised Explosive Devices (IEDs). IEDs are causing injuries in places that are highly placed on the lower extremities in areas such as the femoral artery or junction space. While many new products have been developed to treat hemorrhages, they have been found in practice to be less than ideal in stopping bleeding from IED injuries, and the like, and are generally bulky and take too much time to deploy. For example, due to their bulk, complexity, and lack of ease of application, devices such as (1) SAM® junctional tourniquet, (2) Abdominal Aortic and Junctional Tourniquet (AAJT), and (3) CRoC® junctional tourniquet are not standardly carried in combat medics personal medical bags, and are instead stored in vehicles or helicopters.

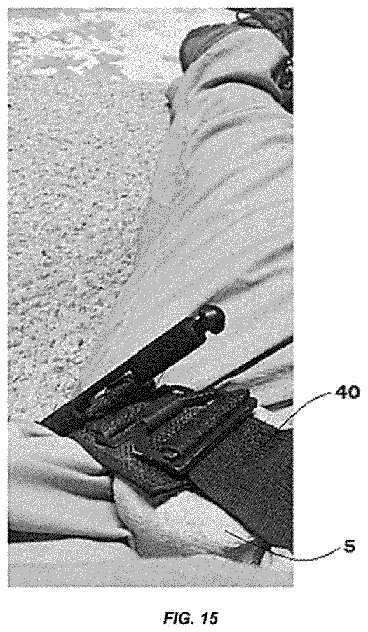

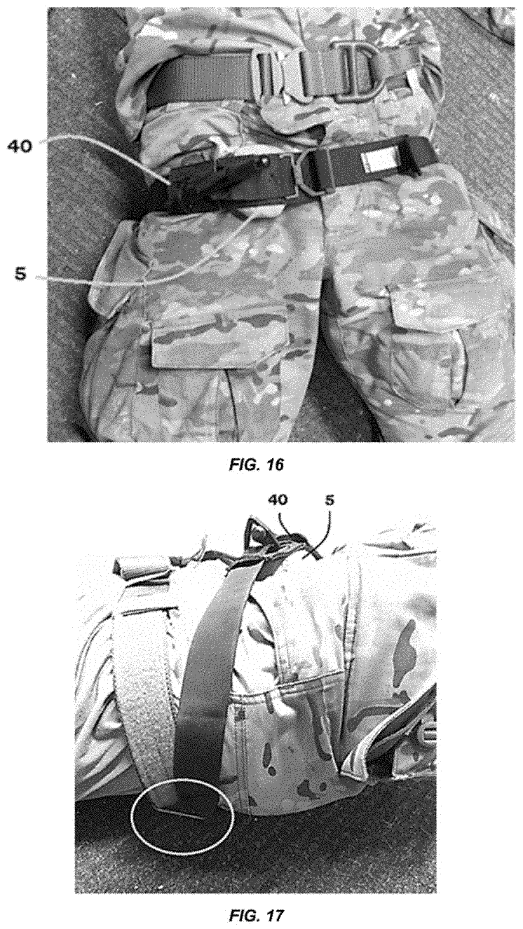

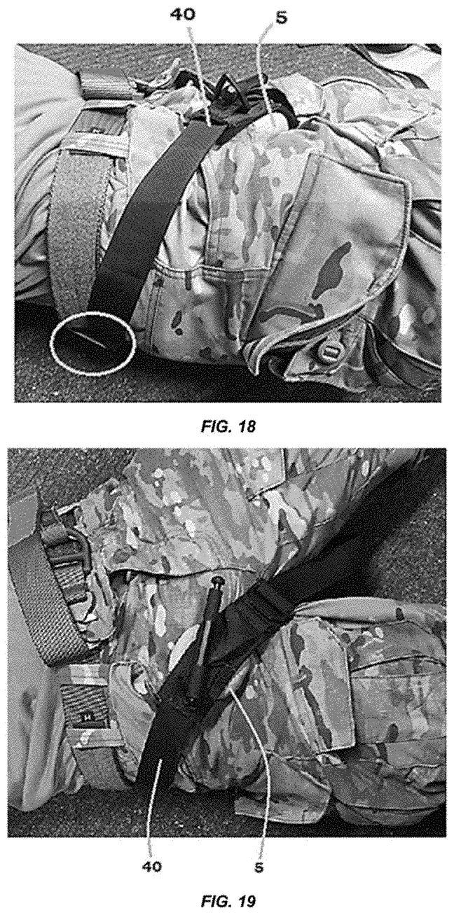

In direct contrast, the junctional hemorrhage control device provided herein is compact, about the size of a hockey puck, which can be easily and speedily applied even by non-combat medics. Due to the device's compact size it can fit into every soldier's individual first aid kit (IFAK). The device provided herein is configured to be used in conjunction with a compressing or constricting device (e.g., a combat tourniquet). Tourniquets and pressure dressings are uniformly and ubiquitously carried by soldiers in order to create hands off, direct pressure to the site of the injury. Moreover, application of the device is facile. For example, when a subject suffers an injury to the femoral artery, then device is positioned above the wound on the upper inner thigh and located over the femoral artery. A tourniquet is applied over the device and tightened. It should be noted, however, the use of the device to control blood flow from an artery and vein is not just limited to the upper inner thigh region. For example, the device can be used with a constricting or compressing in various locations on a subject's body, including the groin region (e.g., see FIG. 16); shoulder region; lower extremities, such as the lower and the upper leg region; and upper extremities, such as the lower arm region and the upper arm region. Moreover, the device can be positioned so as allow for the constricting or compressing device to access a high point of attachment, such as the waist or shoulder (e.g., see FIG. 15). By providing a high point of attachment, the strap or the like of the constricting or compressing device can be inserted through belt loops (e.g., see FIGS. 17-19), wrapped around belts, or inserted through neck and arm openings in shirts, etc. in order to prevent downward/outward displacement of the devices from the original placement point, e.g., when moving the subject or tightening the compressing/constricting device.

The details of one or more embodiments of the disclosure are set forth in the accompanying drawings, as described more fully below.

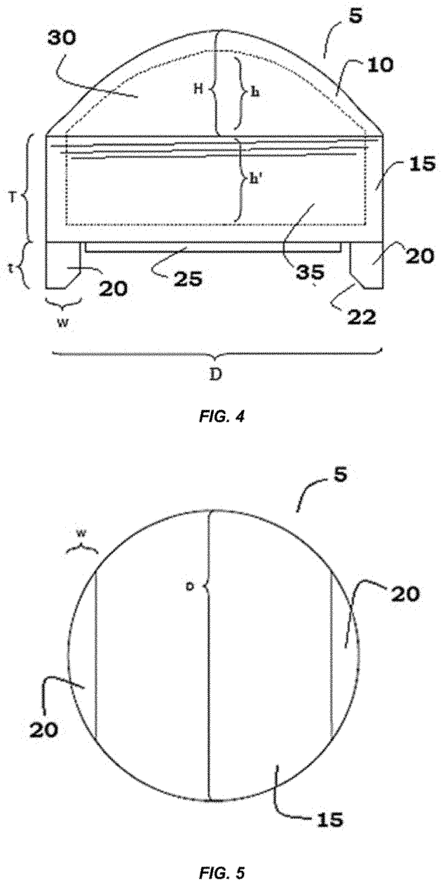

FIG. 3 shows an embodiment of a junctional hemorrhage control device of the disclosure. As shown, the junctional hemorrhage control device 5 comprises a protrusion portion 10 and a base portion 15, wherein protrusion portion 10 is in physical contact with base portion 15. Protrusion portion 10 when connected to base portion 15 may comprise an internal open space within device 5, i.e., device 5 is hollow, or alternatively device 5 may not comprise any open spaces, i.e., device 5 is solid. Protrusion portion 10 may be permanently attached to base portion 15. For example, protrusion portion 10 and base portion 15 can be manufactured as one piece, instead of two separate portions, or alternatively protrusion portion 10 could be glued, welded, cemented, bolted, screwed, riveted or any other similar means of permanent attachment, to base portion 15. Protrusion portion 10 can be made of any suitable lightweight material, including plastic, metal, wood, or combination of any of the foregoing. wherein the protrusion portion 10 should have at least enough tensile strength to resist deforming, breaking, cracking, etc. when pressure is applied to a constricting or compressing device. Base portion 15 can be made of any suitable lightweight material, including plastic, metal, wood, or combination of any of the foregoing. Base portion 15 should have at least enough tensile strength to resist deforming, breaking, cracking, etc. when pressure is applied via tightening a constricting or compressing device. In certain embodiments, protrusion portion 10 and base portion 15 are made from the same material. In alternate embodiments, protrusion portion 10 and base portion 15 are made from different materials. Junctional hemorrhage control device 5 further comprises barrier portion 20. Barrier portion 20 are physically in contact with base portion 15, and are generally in contact with base portion 15 at the edges of base portion 15. While barrier portion 20 are depicted in FIG. 3 as being in physically contact at the outer most edges of base portion 15, barrier portion 20 can also be located so that the outer edges of base portion 15 are not in direct alignment with the outer edges of barrier portion 20, i.e., barrier portion 20 are located not at, but near the outer edges of base portion 15. Barrier portion 20 are typically permanently attached to base portion 15. For example, barrier portion 20 and base portion 15 can be manufactured as one piece, instead of two separate portions, or alternatively barrier portion 20 could be glued, welded, cemented, bolted, screwed, riveted or any other similar means of permanent attachment, to base portion 15. Barrier portion 20 can be made of any suitable lightweight material, including plastic, metal, wood, or combination of any of the foregoing. The interior walls of barrier portion 20 are generally perpendicular to base portion 15 (as shown), but may comprise portions of which that are not perpendicular to base portion 15, i.e., angled >90° to base portion 15 (shown in FIG. 4). Barrier portion 20 are of sufficient height and width apart to allow for a compressing or constriction device to be placed between two barrier portion 20 and prevent the displacement of a constricting or compressing device when the compressing or constricting device is in contact with slippage preventing material/coating 25. In regards to dimensions, protrusion portion 10, at the highest point of curvature from base portion 15 may have a height (H) of at least 0.25, 0.3, 0.375, 0.4, 0.5, 0.6, 0.625, 0.7, 0.75, 0.8, 0.875, 0.9, 1.0, 1.1, 1.125, 1.2, 1.25, 1.3, 1.375, 1.4, or 1.5 inches, or any range between or including any two of the foregoing values. In a particular embodiment, H is about 0.875 inches. Base portion 15 may have a height (T) of at least 0.25, 0.3, 0.375, 0.4, 0.5, 0.6, 0.625, 0.7, 0.75, 0.8, 0.875, 0.9, 1.0, 1.1, 1.125, 1.2, 1.25, 1.3, 1.375, 1.4, or 1.5 inches, or any range between or including any two of the foregoing values. In a particular embodiment, T is about 0.875 inches. Base portion 15 may have a diameter (D) of at least 2, 2.25, 2.3, 2.375, 2.4, 2.5, 2.6, 2.625, 2.7, 2.75, 2.8, 2.875, 2.9, 3.0, 3.1, 3.125, 3.2, 3.25, 3.3, 3.375, 3.4, 3.5, 3.6, 3.7, 3.8, 3.9 or 4 inches, or any range between or including any two of the foregoing values. In a particular embodiment, D is about 2.625 inches. In another embodiment, D is about 3.0 inches. Barrier portion 20 may have a height (t) of at least 0.15, 0.2, 0.25, 0.3, 0.375, 0.4, 0.5, 0.6, 0.625, 0.7, 0.75, 0.8, 0.875, 0.9, or 1.0 inches, or any range between or including any two of the foregoing values. In a particular embodiment, t is about 0.375 inches. Barrier portion 20 may have a width (w) of at least 0.15, 0.2, 0.25, 0.3, 0.375, 0.4, or 0.5 inches, or any range between or including any two of the foregoing values. In a particular embodiment, w is about 0.25 inches or about 0.375 inches.

FIG. 4 provides an alternate embodiment of a junctional hemorrhage control device of the disclosure. As shown, the junctional hemorrhage control device 5 comprises a protrusion portion 10 and a base portion 15, wherein protrusion portion 10 is in physical contact with base portion 15. As shown, protrusion portion 10 may be reversibly attached to base portion 15. For example, protrusion portion 10 and base portion 15 may have matching screw threads so that protrusion portion 10 may be screwed into base portion 15, or vice versa (as shown). Other reversible attachment means may be used, e.g., latches, clips, pins, or the like (not shown). As such, if device 5 comprises an internal open space, reversible attachment of protrusion portion 10 to base portion 15 provides a means to access such open space. According, the open space may be utilized to store various implements and items that can be used with device 5, such as gauze, wrapping, bandages, etc. Alternatively, the open space may be used to store items, implements, etc. that are typically not used with a device, e.g., burn gel, lip balm, etc. In a particular embodiment, accessible open space of device 5 is utilized to store one or more items found in an IFAK kit. The height of open space of protrusion portion 10, at the highest point of curvature from base portion 15 may have a height (h) of at least 0.20, 0.25, 0.3, 0.375, 0.4, 0.5, 0.6, 0.625, 0.7, 0.75, 0.8, 0.875, 0.9, 1.0, 1.1, 1.125, 1.2, 1.25, 1.3, 1.375, or 1.4 inches, or any range between or including any two of the foregoing values. Notably, (h) open space of protrusion portion 10 is less than H of protrusion portion 10. In a particular embodiment, h is from about 0.2 inches to about 0.8 inches. The height of the open space (h′) of base portion 15 is at least 0.20, 0.25, 0.3, 0.375, 0.4, 0.5, 0.6, 0.625, 0.7, 0.75, 0.8, 0.875, 0.9, 1.0, 1.1, 1.125, 1.2, 1.25, 1.3, 1.375, or 1.4 inches, or any range between or including any two of the foregoing values. Notably, (h′) open space of base portion 15 is less than T of base portion 15. In a particular embodiment, h is from about 0.2 inches to about 0.8 inches. Barrier portion 20 may further comprise an internal wall that has a portion that is perpendicular to base portion 15 and a portion of which that is not perpendicular to base portion 15, i.e., angled >90° to base portion 15. The portion that is perpendicular to base portion 15 should have a height that exceeds the thickness of a compressing or constricting device. The portion of which that is not perpendicular to base portion 15, i.e., angled >90° to base portion 15, can be any angle >90°. By barrier portion 20 having an angle (as shown) helps guide the compressing or constricting device over slippage preventing material/coating 25.

FIG. 5 shows a bottoms-up view of a junctional hemorrhage control device of the disclosure. It should be noted at the outset, that although the device is mainly depicted as being generally circular in regards to the shape of base portion 15. Base portion 15 can be any shape, including square, rectangular, oblong, ovoid, irregular, pentagonal, hexagonal, trapezoidal, octahedral, diamond, triangular, etc. Accordingly, D is shown in FIG. 5 and in other Figures, is in general reference to the width, length, or diameter of the device from a point on one edge of base portion 15 to an edge on the directly opposite side of base portion 15. As base portion 15 can have any shape, it should be further recognized that barrier portion 20 is not limited to the segmental shaped pictured, but could also be rectangular, irregular, triangular, etc. in shape. Moreover, as provided above, the outer edges of barrier portion 20 may be congruent to the outer edges of base portion 15 (as shown), or alternatively the outer edges of barrier portion 20 may not be congruent to the outer edges of base portion 15 (not shown). For example, the outer edges of barrier portion 20 may extend from or be within the outer edges of base portion 15 by at least 0.05, 0.1, 0.2, 0.3, 0.4, 0.5, 0.6, 0.7, 0.8, or 0.9 inches, or any range between or including any two of the foregoing values. In further regards to D, in a particular embodiment D is at least 2, 2.25, 2.3, 2.375, 2.4, 2.5, 2.6, 2.625, 2.7, 2.75, 2.8, 2.875, 2.9, 3.0, 3.1, 3.125, 3.2, 3.25, 3.3, 3.375, 3.4, 3.5, 3.6, 3.7, 3.8, 3.9 or 4 inches, or any range between or including any two of the foregoing values.

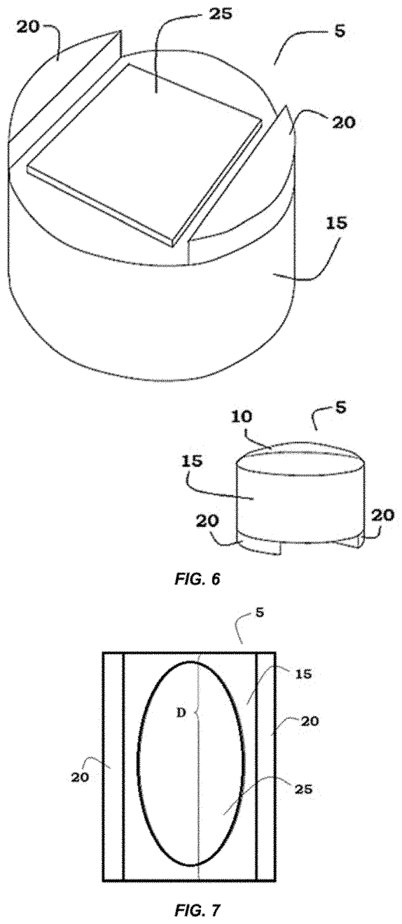

FIG. 6 provides two three-dimensional side angled views of a junctional hemorrhage control device of the disclosure. A three-dimensional side angled close-up view of the base of the junctional hemorrhage control device 5 is provided (top), showing base portion 15, barrier portion 20, and slippage preventing material/coating 25. Also shown is a three-dimension side angled view of the top of the junctional hemorrhage control device 5 (bottom) showing protrusion portion 10, base portion 15, and barrier portion 20. In regards to slippage preventing material/coating 25, slippage preventing material/coating 25 is a mechanical based fastening or dry friction material or dry friction coating on base portion 15 that comes into physical contact with a constricting or compressing device. Slippage preventing material/coating 25 is provided to prevent the displacement or ‘slipping’ of a constricting or compressing device when a constricting or compressing device is used with junctional hemorrhage control device 5. Any type of mechanical based fastening or dry friction material or dry fiction coating can be used for slippage preventing material/coating 25, as long as the material or coating prevents or retards the slippage of a constricting or compressing device when used with device 5. Examples of such mechanical based fastening or dry friction material, include textured or friction fabrics or materials that are adhered or attached to the surface of base portion 15. In regards to coatings, any non-slip, anti-slip, or slip resistant-coating may be used to coat base portion 15 to form slippage preventing material/coating 25. As shown, slippage preventing material/coating 25 can be square shaped, and not full cover the exposed lower surface of base portion 15. It should be understood, however, that the lower surface of base portion 15 may not comprise slippage preventing material/coating 25 at all, or alternatively slippage preventing material/coating 25 may cover a portion of the lower surface of base portion 15 (as shown), or fully cover all of the exposed lower surface (the lower surface portion not covered by barrier portion 20) of base portion 15. For example, a certain portion of the lower surface of base portion 15 may be covered with slippage preventing material/coating 25, e.g., the central portion (as shown), the outer edges, the side edges or a combination of the foregoing. In particular embodiment, slippage preventing material/coating 25 covers at least 10%, 15%, 20%, 25%, 30%, 40%, 50%, 60%, 70%, 80%, or 90% of the lower surface of base portion 15, or any range between or including any two of the foregoing percentages. In another embodiment, slippage preventing material/coating 25 covers the entire (100%) exposed lower surface of base portion 15. In a particular embodiment, protrusion portion 10 can have an arch cross-sectional shape. Examples of arch cross-sectional shapes, include but are not limited to, one-centered (semicircular) (as shown), a two-centered, a three-centered, a four-centered, segmental, a pointed segmental pseudo three-centered, or a pseudo four-centered arch shape.

FIGS. 7-10 provides additional embodiments of alternative shapes and technical features of a junctional hemorrhage control device of the disclosure.

FIG. 7 provides for a bottoms up view of a junctional hemorrhage control device 5 of the disclosure that comprises a base portion 15 that has a rectangular shape. Barrier portion 20 also has a rectangular shape. Slippage preventing material/coating 25 can cover any portion of the lower surface of base portion 15, including the entire lower surface of base portion 15. In a certain embodiment, slippage preventing material/coating 25 can cover multiple noncongruent or congruent portions of the lower surface of base portion 15 (not shown) in a patterned manner or in a non-patterned irregular manner. Examples of patterns can include, but are not limited to, stripes, dots, grids, diamonds, and combinations of the foregoing. Particular examples of patterns that can be used with the slippage preventing material/coating 25 can be found in http://]]--[[gibcoflexmold.com/patterns/, which are incorporated herein by reference. In alternate embodiment, slippage preventing material/coating 25 can cover a single portion of the lower surface of base portion 15 (as shown). Base portion 15 may have a length (D) of at least 2, 2.25, 2.3, 2.375, 2.4, 2.5, 2.6, 2.625, 2.7, 2.75, 2.8, 2.875, 2.9, 3.0, 3.1, 3.125, 3.2, 3.25, 3.3, 3.375, 3.4, 3.5, 3.6, 3.7, 3.8, 3.9 or 4 inches, or any range between or including any two of the foregoing values. In a particular embodiment, base portion 15 has a length of about 2.625 inches. In a further embodiment, base portion 15 has a width of about 2.625 inches. Barrier portion 20 may have a width of at least 0.15, 0.2, 0.25, 0.3, 0.375, 0.4, or 0.5 inches, or any range between or including any two of the foregoing values. In a particular embodiment, barrier portion 20 has a width of about 0.375 inches.

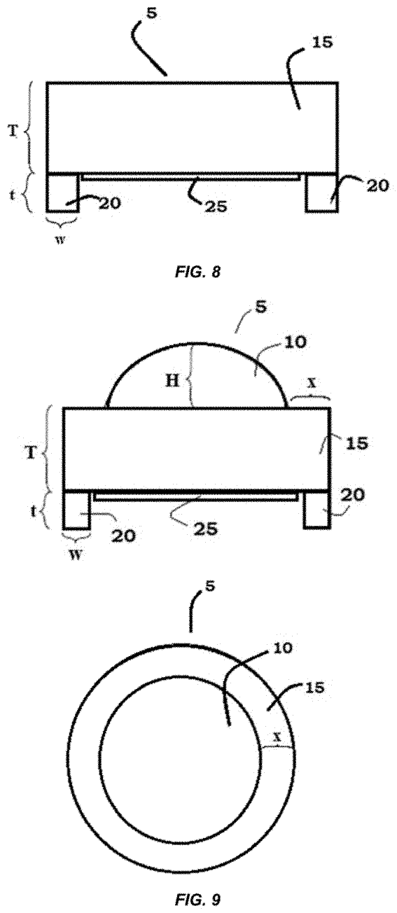

FIG. 8 provides a front on side view of a junctional hemorrhage control device 5 of the disclosure that comprises base portion 15, barrier portion 20 and slippage preventing material/coating 25. Notably, device 5 is shown as not comprising protrusion 10. Accordingly, in certain embodiments, device 5 may not comprise protrusion portion 10. In alternate embodiments, device 5 may not comprise barrier portion 20. In further embodiments, device 5 may not comprise barrier portion 20 and protrusion portion 10. T, t, and w can have the dimensions already expressed herein.

FIG. 9 provides two views, a front on side view (top) and a top down view (bottom), of an embodiment of a junctional hemorrhage control device 5, wherein protrusion portion 10 is convex shaped and has edges that are not congruent with the outer edges of base portion 15. Device 5 further comprises barrier portion 20 and slippage preventing material/coating 25. In a particular embodiment, the distance between the outer edge of base portion to the outer edge of protrusion 10 is designated by X, wherein X is at least 0.05, 0.1, 0.2, 0.3, 0.4, 0.5, 0.6, 0.7, 0.8, 0.9, 1.0, 1.1, 1.2, 1.3, 1.4 or 1.5 inches, or any range between or including any two of the foregoing values. T, t, and w can have the dimensions already expressed herein.

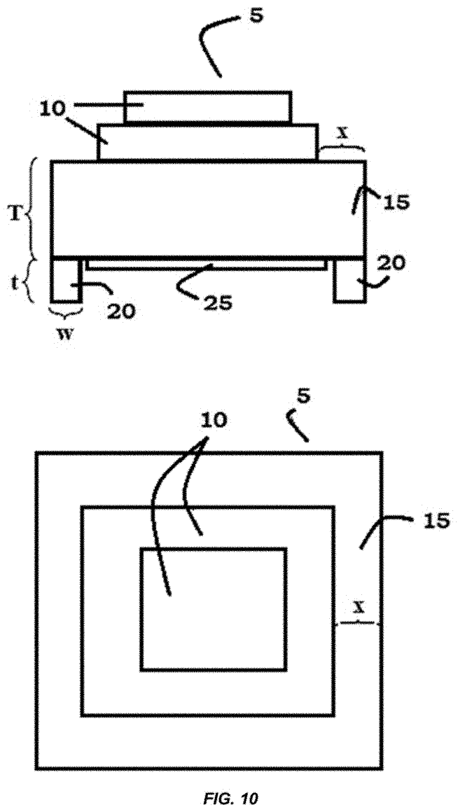

FIG. 10 provides two views, a front on side view (top) and a top down view (bottom), of an embodiment of a junctional hemorrhage control device 5, wherein protrusion portion 10 comprises multiple layers that have edges which are not congruent with the outer edges of base portion 15. In regards to the layers of protrusion portion 10, generally, going from the base portion 15 outwards, each layer should have smaller dimensions (as shown). However, it should be understood that outer layers need not have smaller dimensions than preceding layers, and can have any shape in addition to the cuboidal shape as shown. For example, the bottom layers may be generally cylindrical, while the outer layers may be cuboidal, convex shaped or have another shape. Alternatively, the bottom layers may be cuboidal, while the outer layers may be convex shaped, cylindrical, or have another shape. As shown, device 5 further comprises barrier portion 20 and slippage preventing material/coating 25. In a particular embodiment, the distance between the outer edge of base portion to the outer edge of lowest layer of protrusion portion 10 is designated by X, wherein X is at least 0.05, 0.1, 0.2, 0.3, 0.4, 0.5, 0.6, 0.7, 0.8, 0.9, 1.0, 1.1, 1.2, 1.3, 1.4 or 1.5 inches, or any range between or including any two of the foregoing values. T, t, and w can have the dimensions already expressed herein.

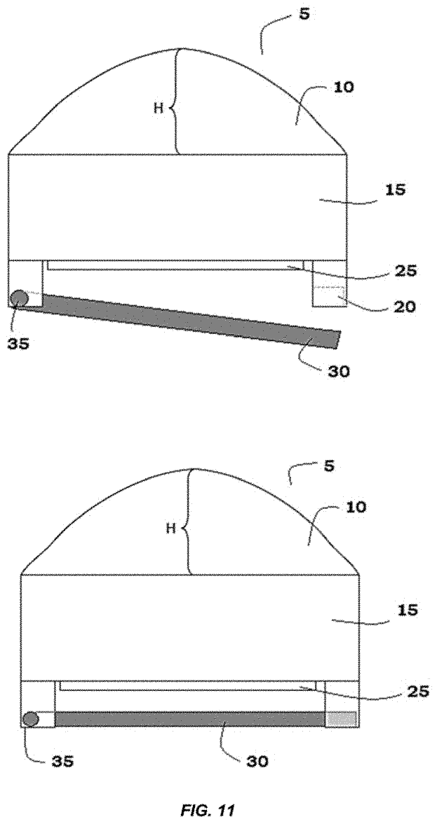

FIG. 11 provides two views of an embodiment of a junctional hemorrhage control device 5 which comprises protrusion 10, base portion 15, barrier portion 20, slippage preventing material/coating 25, latch 30 and hinge 35. In the top view, latch 30 is in an open position, while in the lower view, latch 30 is in a closed position. Hinge 35 provides a means to swing latch 30 from an open to closed position, and vice versa. Hinge 35 can be fixably attached to barrier portion 25. Latch 30 when attached to hinge 35 can move in an angle from 0 degrees to 15, 30, 45, 50, 60, 75, 90 degrees, or greater than 90 degrees. In a particular embodiment, one end of latch 30 is attached to hinge 35, where the other end can slideably insert into a notch provided on barrier portion 20, wherein the latch 30 can be fixed in place using a fixing means so that the latch cannot be moved from a closed position without using an opening means. In alternate embodiments, latch 30 may slide into notches in both barrier portion 20 and not be attached to a hinge 35. In such embodiments, latch 30 is fixed in place in the notches of barrier 20 using a fixing means so that the latch cannot be moved from a closed position without using an opening means. A fixing means, can include, but not limited to, latches, clasps, clips, and the like. An opening means, can include, but not limited to, releases, applying pressure, etc. Latch 30 is configured to help maintain the connection between a constricting or compressing device to slippage preventing material/coating 25 of a junctional hemorrhage control device 5. Accordingly, the space between latch 30 and slippage preventing material/coating 25 should be able to accommodate a constricting or compressing device.

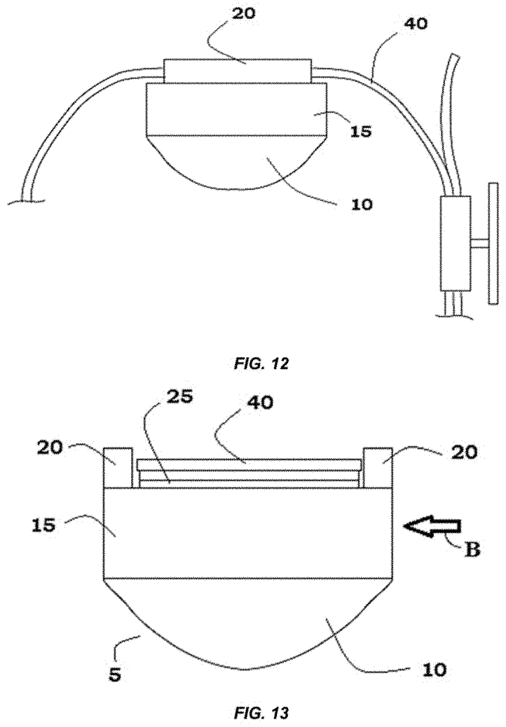

FIG. 12 provides a side on view of an embodiment of a junctional hemorrhage control device 5 being used with a constricting or compressing device 40. Constricting or compressing device 40 is pressed into contact with slippage preventing material/coating 25 (not shown), and barrier portion 20 prevents the displacement of the constricting or compressing device 40 from junctional hemorrhage control device 5. Protrusion 10 is applied against a location on a subject's body, preferably over an artery and/or vein, and then the constricting or compressing device 40 is tightened, so as to control venous and arterial circulation to an extremity for a period of time. Examples of constricting or compressing device 40, include, but are not limited to, tourniquets, straps, bandages, belts, cravats, flattened hoses, tubing, and the like. In a particular embodiment, constricting or compressing device 40 is a tourniquet or pressure bandage.

FIG. 13 provides another side on view of an embodiment of a junctional hemorrhage control device 5 of the disclosure being used with a constricting or compressing device 40. As shown, constricting or compressing device 40 comes into direct contact with slippage preventing material/coating 25 and fits with the cavity formed between barrier portion 20. In further embodiments, latch 30 can be affixed to the device 5, so as to prevent the displacement of constricting or compressing device 40 from device 5.

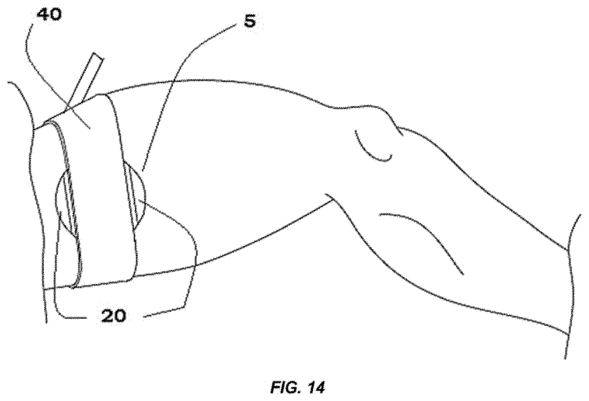

FIG. 14 provides a view of a junctional hemorrhage control device 5 of the disclosure being used with a constricting or compressing device 40 on the thigh of a subject. As shown, barrier portion 20 prevents the slippage of constricting or compressing device 40 from the junctional hemorrhage control device 5. Protrusion 10 is placed against the leg (not shown) and is located over the femoral artery and vein. Accordingly, tightening constricting or compressing device 40 puts pressure on the junctional hemorrhage control device 5, where the pressure exerted via protrusion 10 controls the blood flow through the femoral artery and vein.

FIG. 15 provides a picture of a junctional hemorrhage control device 5 of the disclosure being used with a constricting or compressing device 40 on the thigh of a subject.

FIG. 16 provides a picture of a junctional hemorrhage control device 5 of the disclosure being used with a constricting or compressing device 40, where the devices are placed around the groin region of a subject.

FIGS. 17-19 provides pictures of various views of a junctional control device 5 of the disclosure being used with a constricting or compressing device 40 on the upper inner thigh region of a subject. A high placement point of the devices allows for access to belts, belt loops (as shown), shirt openings, etc. thereby providing a means to stabilize the placement point of the devices from unwanted downward/outward movement.

It will be understood that various modifications may be made without departing from the spirit and scope of the invention. Other embodiments are within the scope of the following claims.

1. A junctional hemorrhage control device comprising:
a protrusion portion that has a generally rounded upper surface and a flat bottom lower surface;
a barrier portion comprising a left portion and a right portion, wherein the left portion and the right portion have the same geometries and dimensions;
a base portion, wherein the flat bottom surface of the protrusion portion is in physical contact and attached to the base portion, and wherein the highest point of curvature for the upper surface of the protrusion portion is located above the center point of the base portion, and wherein the left portion and right portion of the barrier portion are in physical contact and attached to opposite sides of the lower surface of the base portion so as extend from opposite ends of the base portion, wherein between the left portion and right portion is a rectangular space; and
a dry friction coating or material that is physically in contact with the central lower surface of the base portion and located in the rectangular space between the left portion and right portion of the barrier portion;
wherein the junctional hemorrhage control device is configured to be used with a compressing or constricting device to control the blood flow of an artery and/or vein in an extremity of a subject.

2. The junctional hemorrhage control device of claim 1, wherein a cross-section section of the protrusion portion has an arch-like shape.

3. The junctional hemorrhage control device of claim 2, wherein the arch-like shape is selected from the group consisting of one-centered (semicircular), two-centered, three-centered, four-centered, segmental, pointed segmental pseudo three-centered, or pseudo four-centered.

4. The junctional hemorrhage control device of claim 1, wherein the edges of the protrusion portion are congruent with the edges of the base portion.

5. The junctional hemorrhage control device of claim 1, wherein the edges of the protrusion portion are not congruent with the edges of the base portion, wherein the distance between the outer edge of base portion to the outer edge of protrusion portion is from 0.05 to 0.5 inches.

6. The junctional hemorrhage control device of claim 1, wherein the highest point of curvature from lower surface of the protrusion portion is from 0.5 to 1.125 inches.

7. The junctional hemorrhage control device of claim 1, wherein the protrusion portion is permanently attached to the base portion.

8. The junctional hemorrhage control device of claim 1, wherein the protrusion portion is reversibly attached to the base portion, and wherein the protrusion portion and base portion comprise an inner open space so that when protrusion portion and base portion are attached, the junctional hemorrhage control device is hollow.

9. The junctional hemorrhage control device of claim 1, wherein the base portion has a diameter or length from 2.5 to 4 inches, and a height of 0.5 to 1.2 inches.

10. The junctional hemorrhage control device of claim 1, wherein the base portion has a cylindrical shape, and the left portion and right portion of the barrier portion have segmented shapes.

11. The junctional hemorrhage control device of claim 1, wherein the width of the left and right portions of the barrier portion is from 0.15 to 0.4 inches.

12. The junctional hemorrhage control device of claim 1, wherein the base portion has a diameter of about 2.625 inches or about 3 inches and the width of the left and right portion of the barrier portion is about 0.25 inches or about 0.375 inches.

13. The junctional hemorrhage control device of claim 1, wherein the dry friction coating or material is a non-slip, anti-slip, or slip resistant-coating material that is applied to the surface of the base portion.

14. The junctional hemorrhage control device of claim 1, wherein the left portion and the right portion of the barrier portion further comprises notches; and wherein the junctional hemorrhage control device further comprises a latch that can slideably fit within the notches of the left and right portions and that can be fixed in place by using a fixing means, and which can be released from the notches by using an opening means.

15. A method of using the junctional hemorrhage control device of claim 1 comprising:
contacting the protrusion portion of the junctional hemorrhage control device with an extremity of a subject, wherein the junctional hemorrhage control device is placed over an artery and/or vein;
affixing a compressing or constriction device around the extremity and in contact with the dry friction coating or material on the base portion of the junctional hemorrhage control device; and
tightening the compressing or constriction device so as to control the blood flow through the artery and or vein of the extremity of the subject.

16. The method of claim 15, wherein the junctional hemorrhage control device is placed over the femoral artery and vein of the thigh portion of the lower extremity of the subject.

17. The method of claim 15, wherein the compressing or constriction device is a tourniquet.
